(19) United States
(12) Patent Application Publication
(10) Pub. No.: US 2004/0180862 A1
(43) Pub. Date: Sep. 16, 2004

(54) Compositions and methods for inhibiting bone resorption

(76) Inventors: Alfred A. Reszka, Glenside, PA (US); John E. Fisher, Jenkintown, PA (US)

Correspondence Address: MERCK AND CO INC, P O BOX 2000, RAHWAY, NJ, 070650907

(21) Appl. No.: 10/479,922

(22) PCT Filed: May 31, 2002
(86) PCT No.: PCT/US02/17142

(30) Foreign Application Priority Data
Jun. 5, 2001 (US) 60295876

(51) Int. Cl. A61K 31/675; A61K 31/663

(52) U.S. Cl. 514/89; 514/102

(57) ABSTRACT

The present invention relates to parenteral compositions and methods for inhibiting bone resorption in a mammal while counteracting potential local irritation at injection sites. The compositions useful herein comprise the combination of a pharmaceutically effective amount of a farnesyl diphosphate synthase inhibiting bisphosphonate or a pharmaceutically-acceptable salt thereof and a pharmaceutically effective amount of a squalene synthase inhibitor.

### BRIEF DESCRIPTION OF THE INVENTION

The present invention relates to parenteral compositions and methods for inhibiting bone resorption in a mammal while counteracting potential local irritation at injection sites. The compositions useful herein comprise the combination of a pharmaceutically effective amount of a farnesyl diphosphate synthase inhibiting bisphosphonate or a pharmaceutically-acceptable salt thereof and a pharmaceutically effective amount of a squalene synthase inhibitor.

### BACKGROUND OF THE INVENTION

A variety of disorders in humans and other mammals involve or are associated with abnormal bone resorption. Such disorders include, but are not limited to, osteoporosis, Paget's disease, periprosthetic bone loss or osteolysis, and hypercalcemia of malignancy. The most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are a major cause of morbidity and mortality in the elderly population. As many as 70% of women and a third of men will experience an osteoporotic fracture. A large segment of the older population already has low bone density and a high risk of fractures. There is a significant need to both prevent and treat osteoporosis and other conditions associated with bone resorption. Because osteoporosis, as well as other disorders associated with bone loss, are generally chronic conditions, it is believed that appropriate therapy will typically require chronic treatment.

Multinucleated cells called osteoclasts are responsible for causing bone loss through a process known as bone resorption. It is well known that bisphosphonates are selective inhibitors of osteoclastic bone resorption, making these compounds important therapeutic agents in the treatment or prevention of a variety of generalized or localized bone disorders caused by or associated with abnormal bone resorption. See H. Fleisch, Bisphosphonates In Bone Disease, From The Laboratory To The Patient, 4th Edition, Academic Press (2000), which is incorporated by reference herein in its entirety. Without being limited by theory, it is believed that bisphosphonates inhibit osteoclast function by triggering apoptosis, i.e. programmed cell death. See D. E. Hughes et al., “Bisphosphonates promote apoptosis in murine osteoclasts in vitro and in vivo”, Journal of Bone and Mineral Research, 10 (10), 1478-1487 (1995), which is incorporated by reference herein in its entirety.

At present, a great amount of preclinical and clinical data exists for the potent nitrogen-containing bisphosphonate compound, alendronate. Evidence suggests that other farnesyl diphosphate synthase inhibiting bisphosphonates such as pamidronate, risedronate, ibandronate, incadronate, olpadronate and zolendronate, have many properties in common with alendronate, including high potency as inhibitors of osteoclastic bone resorption.

Despite their therapeutic benefits, bisphosphonates are poorly absorbed from the gastrointestinal tract. See B. J. Gertz et al., Clinical Pharmacology of Alendronate Sodium, Osteoporosis Int., Suppl. 3: S13-16 (1993) and B. J. Gertz et al., Studies of the oral bioavailability of alendronate, Clinical Pharmacology & Therapeutics, vol. 58, number 3, pp. 288-298 (September 1995), which are incorporated by reference herein in their entirety. Intravenous administration has been used to overcome this bioavailability problem. However, intravenous administration is costly and inconvenient, especially when the patient must be given an intravenous infusion lasting several hours on repeated occasions.

A quicker and simpler means of administration could be achieved through alternate means of parenteral administration. The patient could perform the injection at home, much like with insulin, thus avoiding the need for costly and time-consuming visits to the doctor's office or clinic. However, the potent bisphosphonates are also known to irritate the stratified squamous epithelium, such as lines the esophagus or comprises the upper layers of the dermis.

In the present invention, the combination of a farnesyl diphosphate synthase inhibiting bisphosphonate or a pharmaceutically-acceptable salt thereof and a squalene synthase inhibitor is highly effective for inhibiting bone resorption while mitigating the potential local irritation at injection sites that can be associated with bisphosphonate therapy. The combination has the advantage of providing increased safety and better patient compliance, which should maximize therapeutic efficacy. It is believed that the squalene synthase inhibitor blocks the potentially harmful effect of the bisphosphonate on the epithelial cells of the skin by causing accumulation of protein isoprenylation precursors, which when added exogenously block the induction of apoptosis by the farnesyl diphosphate synthase inhibiting bisphosphonates. By selecting an appropriate dosage of the squalene synthase inhibitor it is possible to parenterally deliver a sufficiently high local concentration of the squalene synthase inhibitor to the desired site to block the potentially harmful effects of the farnesyl diphosphate synthase inhibiting bisphosphonate, while minimizing the blocking effect on the osteoclasts, where the full therapeutic benefit of the bisphosphonate is desired to inhibit bone resorption.

It is an object of the present invention to provide compositions comprising the combination of a farnesyl diphosphate synthase inhibiting bisphosphonate or a pharmaceutically-acceptable salt thereof and a squalene synthase inhibitor.

It is another object of the present invention to provide improved parenteral methods for inhibiting bone resorption and the conditions associated therewith in a mammal, particularly wherein said mammal is a human.

It is another object of the present invention to provide improved parenteral methods for treating or preventing abnormal bone resorption and the conditions associated therewith.

It is another object of the present invention to provide such parenteral methods while counteracting potential adverse gastrointestinal effects.

It is another object of the present invention to provide such methods wherein the dosing is maintained until the desired therapeutic effect is achieved.

It is another object of the present invention to treat or prevent abnormal bone resorption in an osteoporotic mammal, preferably an osteoporotic human.

These and other objects will become readily apparent from the detailed description which follows.

### SUMMARY OF THE INVENTION

The present invention relates to a pharmaceutical composition comprising a farnesyl diphosphate synthase inhibiting bisphosphonate or pharmaceutically acceptable salt thereof and a squalene synthase inhibitor.

In further embodiments, the present invention relates to a pharmaceutical composition comprising a pharmaceutically-effective amount of a farnesyl diphosphate synthase inhibiting bisphosphonate or pharmaceutically acceptable salt thereof and an amount of a squalene synthase inhibitor effective to counteract farnesyl diphosphate synthase inhibiting bisphosphonate-associated local irritation at injection sites.

In further embodiments, the present invention relates to a method for inhibiting bone resorption in a mammal in need thereof comprising administering a farnesyl diphosphate synthase inhibiting bisphosphonate or pharmaceutically acceptable salt thereof and a squalene synthase inhibitor.

In further embodiments, the present invention relates to a method for inhibiting bone resorption in a mammal in need thereof comprising sequentially administering a squalene synthase inhibitor and a nitrogen-containing bisphosphonate or pharmaceutically acceptable salt thereof.

In further embodiments, the present invention relates to the use of a composition in the manufacture of a medicament for inhibiting bone resorption in a mammal in need thereof, said composition comprising a farnesyl diphosphate synthase inhibiting bisphosphonate or pharmaceutically acceptable salt thereof and a squalene synthase inhibitor.

In further embodiments, the present invention relates to the use of a composition comprising a farnesyl diphosphate synthase inhibiting bisphosphonate or pharmaceutically acceptable salt thereof and a squalene synthase inhibitor for inhibiting bone resorption in a mammal in need thereof.

All percentages and ratios used herein, unless otherwise indicated, are by weight. The invention hereof can comprise, consist of, or consist essentially of the essential as well as optional ingredients, components, and methods described herein.

### BRIEF DESCRIPTION OF THE FIGURES

FIG. 1

Squalene synthase inhibitor suppression of the inhibition of protein isoprenylation induced in Ch1.Es esophageal fibroblasts by alendronate.

Ch1.Es fibroblasts were grown in culture for 24 hours prior to treatment with alendronate [ALN] at 300 μM. Cells were treated for 24 hours and protein lysates were analyzed for suppression of protein isoprenylation. Markers included either anti-hDNAJ, a farnesylated protein that migrates more slowly when farnesylation is absent (two bands observed) or anti-Rap1A, which we have shown previously to bind preferentially to Rap1A if isoprenylation is blocked (presence of a band indicates absence of protein geranylgeranylation) (See Reszka, A. A., Halasy-Nagy, J. and Rodan, G. A. Molecular Pharmacology 59:193-202, 2001).

(A) Cells pretreated for 24 hours with nothing or the squalene synthase inhibitor, zaragozic Acid A (Zara-A) at 3 μM (three lanes indicated). Cells were subsequently treated with alendronate (ALN, 300 μM) or a geranylgeranyl transferase inhibitor (GGTI, 0.1 μM). Note that ALN, but not GGTI, inhibition of Rap1A geranylgeranylation (manifest as presence of the dark band in the immunoblot) is diminished when cells are pretreated with Zara-A.

(b) Cells were pretreated for 24 hours with varying concentrations of Zara-A (0 and 0.1-10 μM) and then continued for another 24 hours in the absence or presence of ALN. Note that Zara-A dose-dependently diminished the capacity of ALN to inhibit hDNAJ farnesylation (inhibition being manifest as the formation of a dark band above the hDNAJ band seen without any treatment) and Rap1A geranylgeranylation.

FIG. 2

Squalene synthase inhibitor suppression of the induction of apoptosis induced in Ch1.Es esophageal fibroblasts by ALN.

Ch1.Es cells were grown for 24 hours in the absence (top and bottom panels) or presence (middle) of Zara-A. Continuing for an additional 24 hours, cells were treated without bisphosphonate (No BP), with ALN (300 μM) or with ALN and geranylgeraniol (GGOH) at 10 μM. Apoptosis is observed as cell rounding and detachment from the dish.

FIG. 3

A squalene synthase inhibitor in the absence of lipoprotein maximally suppresses alendronate and risedronate-induced apoptosis.

Ch1.Es cells were grown for 24 hours in either fetal bovine serum (FBS) (top panels) or lipoprotein-deficient PBS (LPDS) in the absence (middle) or presence (bottom) of Zara-A (10 μM). Continuing for an additional 24 hours, cells were treated without bisphosphonate (No BP), with ALN (300 μM) or risedronate [RIS] (300 μM). Phenotypes were scored as in FIG. 2. The suppression of apoptosis is near complete after inclusion of Zara-A.

FIG. 4

ALN and RIS activate pro-apoptotic kinases in Ch1.Es esophageal fibroblasts.

Cells were grown for 24 hours in the presence of vehicle, ALN (300 μM), RIS (300 μM), the HMG-CoA reductase inhibitor lovastatin (10 μM), farnesyl transferase inhibitor (FTI, 100 nM) or geranylgeranyl transferase inhibitor (GGTI, 100 nM). Cell lysates were analyzed using an in-gel kinase assay (top) or by immuno-blotting with anti-phospho-specific antibodies recognizing active MAP kinases (Erk1 and 2), p38, or Jun N-terminal kinase (Jnk).

FIG. 5

Skin irritation induced by alendronate and risedronate is mimicked by an inhibitor of HMG CoA-reductase in vivo.

Rats were injected subcutaneously for a period of 5 days (vehicle, ALN [0.5 mg/kg] or RIS [0.5 mg/kg]) or 2 days (vehicle or simvastatin [SIM] at 10 mg/kg). Skin was harvested after necropsy, fixed with formalin, thin-sectioned and stained with hematoxylin and eosin. Area was determined by measuring the thickness and length of each affected area and is represented vs. the H₂O control.

FIG. 6

A squalene synthase inhibitor suppresses ALN-induced skin irritation in vivo.

Rats were injected subcutaneously for a period of 5 days with H₂O vehicle, ALN [0.5 mg/kg, ALN with Zara-A (3 μg/kg) or with Zara-A alone. Samples were processed as in FIG. 4. The inverted “T” is used to show the thickness and length of the area affected by ALN.

FIG. 7

A squalene synthase inhibitor suppresses ALN- or RIS-induced skin irritation in vivo.

Rats were injected subcutaneously for a period of 5 days with H₂O vehicle, ALN or RIS (0.5 mg/kg), ALN or RIS with Zara-A (3 μg/kg) or with Zara-A alone. Skin was harvested after necropsy and the affected area was measured for thickness and diameter using a caliper micrometer. Affected areas were circular in shape and thus volumetric analyses were determined by multiplying the area of the circle by the thickness. Note that while volume ranged from 50-100 mm³ in the vehicle control, it ranged from 240-280 after treatment with RIS or ALN, respectively. Coadministration with Zara-A significantly reduced thickening induced by either ALN or RIS (P≦0.01).

### DETAILED DESCRIPTION OF THE INVENTION

The present invention relates to compositions and methods for inhibiting bone resorption in a mammal in need of such treatment, while counter-acting the occurrence of local irritation at injection sites. The compositions comprise a pharmaceutically effective amount of a farnesyl diphosphate synthase inhibiting bisphosphonate or a pharmaceutically-acceptable salt thereof and a pharmaceutically effective amount of a squalene synthase inhibitor.

The term “pharmaceutically effective amount”, as used herein, means that amount of the farnesyl diphosphate synthase inhibiting bisphosphonate compound or squalene synthase inhibitor, that will elicit the desired therapeutic effect or response or provide the desired benefit when administered in accordance with the desired treatment regimen. A preferred pharmaceutically effective amount of the farnesyl diphosphate synthase inhibiting bisphosphonate is a bone resorption inhibiting amount. A preferred pharmaceutically effective amount of the squalene synthase inhibitor is an amount that will counteract, i.e. block or mitigate, the occurrence of local irritation at injection sites, while not counteracting, or only minimally counter-acting, the therapeutic bone resorption effects of the farnesyl diphosphate synthase inhibiting bisphosphonate.

The term “counteracting the occurrence of local irritation at injection sites”, as used herein, means to prevent, block, decrease, or lessen the occurrence of unwanted side effects local irritation at injection sites, relative to treatment with a farnesyl diphosphate synthase inhibiting bisphosphonate alone.

In the present invention it is an object to inhibit bone resorption, or more specifically to inhibit undesired or abnormal bone resorption. The term “abnormal bone resorption”, as used herein means a degree of bone resorption that exceeds the degree of bone formation, either locally, or in the skeleton as a whole. Alternatively, “abnormal bone resorption” can be associated with the formation of bone having an abnormal structure, as in Paget's disease.

The term “bone resorption inhibiting”, as used herein, means preventing bone resorption by the direct or indirect alteration of osteoclast formation or activity. Inhibition of bone resorption refers to prevention of bone loss, especially the inhibition of removal of existing bone either from the mineral phase and/or the organic matrix phase, through direct or indirect alteration of osteoclast formation or activity.

The term “until the desired therapeutic effect is achieved”, as used herein, means that the therapeutic agent or agents are continuously administered, according to the dosing schedule chosen, up to the time that the clinical or medical effect sought for the disease or condition being treated is observed by the clinician or researcher. For methods of treatment of the present invention, the pharmaceutical composition is continuously administered until the desired change in bone mass or structure is observed. In such instances, achieving an increase in bone mass or a replacement of abnormal bone structure with normal bone structure are the desired objectives. For methods of prevention of the present invention, the pharmaceutical composition is continuously administered for as long as necessary to prevent the undesired condition. In such instances, maintenance of bone mass density is often the objective. Nonlimiting examples of administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

The term “farnesyl diphosphate synthase inhibiting” as used herein means that the bisphosphonate in question acts at the molecular level to suppress farnesyl diphosphate synthase and, as such, attains its anti-resorptive activity through this molecular action. For example, alendronic acid, i.e. 4-amino-1-hydroxybutylidene-1,1-bisphosphonic acid is an example of a farnesyl diphosphate synthase inhibiting bisphosphonate.

The term “parenteral administration” as used herein means taken into the body or administered in a manner other than through the digestive tract, as by intravenous, subcutaneous or intramuscular injection.

Compositions of the Present Invention

The pharmaceutical compositions of the present invention comprise a pharmaceutically effective amount of a farnesyl diphosphate synthase inhibiting bisphosphonate or a pharmaceutically-acceptable salt thereof and a pharmaceutically effective amount of a squalene synthase inhibitor. These compositions are useful for inhibiting bone resorption in a mammal in need thereof while counteracting the potentially adverse effects, such as local irritation at injection sites, that can be associated with the parenteral administration of the bisphosphonate.

Farnesyl Diphosphate Synthase Inhibiting Bisphosphonates

The methods and compositions of the present invention comprise the administration of a farnesyl diphosphate synthase inhibiting bisphosphonate or a pharmaceutically acceptable salt thereof. The farnesyl diphosphate synthase inhibiting bisphosphonates of the present invention correspond to the chemical formula

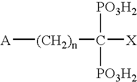

wherein n is an integer from 0 to 7 and wherein A and X are independently selected from the group consisting of H, OH, halogen, NH₂, SH, phenyl, C₁-C₃₀ alkyl, C₃-C₃₀ branched or cycloalkyl, C₁-C₃₀ substituted alkyl, C₁-C₁₀ alkyl substituted NH₂, C₃-C₁₀ branched or cycloalkyl substituted NH₂, C₁-C₁₀ dialkyl substituted NH₂, C₁-C₁₀ alkoxy, C₁-C₁₀ alkyl substituted thio, thiophenyl, halophenylthio, C₁-C₁₀ alkyl substituted phenyl, pyridyl, furanyl, pyrrolidinyl, imidazolyl, imidazopyridinyl, and benzyl, such that both A and X are not selected from H or OH when n is 0; or A and X are taken together with the carbon atom or atoms to which they are attached to form a C₃-C₁₀ ring.

In the foregoing chemical formula, the alkyl groups can be straight, branched, or cyclic, provided sufficient atoms are selected for the chemical formula. The C₁-C₃₀ substituted alkyl can include a wide variety of substituents, nonlimiting examples which include those selected from the group consisting of phenyl, pyridyl, furanyl, pyrrolidinyl, imidazonyl, NH₂, C₁-C₁₀ alkyl or dialkyl substituted NH₂, OH, SH, and C₁-C₁₀ alkoxy.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

A non-limiting class of structures useful in the instant invention are those in which A is selected from the group consisting of H, OH, and halogen, and X is selected from the group consisting of C₁-C₃₀ alkyl, C₁-C₃₀ substituted alky, halogen, and C₁-C₁₀ alkyl or phenyl substituted thio.

A non-limiting subclass of structures useful in the instant invention are those in which A is selected from the group consisting of H, OH, and Cl, and X is selected from the group consisting of C₁-C₃₀ alky, C₁-C₃₀ substituted alkyl, Cl, and chlorophenylthio.

A non-limiting example of the subclass of structures useful in the instant invention is when A is OH, X is a 3-aminopropyl moiety and n is zero, so that the resulting compound is a 4-amino-1,1-hydroxybutylidene-1,1-bisphosphonate, i.e. alendronate.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C₁-C₃₀-alkyl-substituted ammonium. Preferred salts are those selected from the group consisting of sodium, potassium, calcium, magnesium, and ammonium salts. More preferred are sodium salts. Non-imiting examples of derivatives include those selected from the group consisting of esters, hydrates, and amides.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those of ordinary skill in the art, reference to a specific weight or percentage of a bisphosphonate compound in the present invention is on an acid active weight basis, unless indicated otherwise herein. For example, the phrase “about 5 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 5 mg of alendronic acid.

Non-limiting examples of bisphosphonates useful herein include the following:

Alendronic acid, 4-amino-1-hydroxybutylidene-1,1-bisphosphonic acid.

Alendronate (also known as alendronate sodium or alendronate monosodium trihydrate), 4-amino-1-hydroxybutylidene-1,1-bisphosphonic acid monosodium trihydrate.

Alendronic acid and alendronate are described in U.S. Pat. No. 4,922,007, to Kieczykowski et al., issued May 1, 1990; U.S. Pat. No. 5,019,651, to Kieczykowski et al., issued May 28, 1991; U.S. Pat. No. 5,510,517, to Dauer et al., issued Apr. 23, 1996; U.S. Pat. No. 5,648,491, to Dauer et al., issued Jul. 15, 1997, all of which are incorporated by reference herein in their entirety.

Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem. 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

1-hydroxy-2-imidazo-(1,2-a)pyridin-3-yethylidene (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate).

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zoledronate).

A non-limiting class of bisphosphonates useful in the instant invention are selected from the group consisting of alendronate, cimadronate, clodronate, etidronate, ibandronate, incandronate, minodronate, neridronate, olpadronate, pamidronate, piridronate, risedronate, tiludronate, zoledronate, pharmaceutically acceptable salts thereof, and mixtures thereof.

More preferred is alendronate, ibandronate, risedronate, pharmaceutically acceptable salts or esters thereof, and mixtures thereof.

A non-limiting subclass of the above-mentioned class useful in the instant case contains alendronate, pharmaceutically acceptable salts thereof, esters thereof and mixtures thereof.

A nonlimiting subclass of the above-mentioned class useful in the instant case is such that the pharmaceutically acceptable salts of alendronate are selected from the group consisting of sodium, potassium calcium, magnesium, and ammonium salts. In a further nonlimiting class the salts are sodium salts, nonlimiting examples of which include for example, the monosodium, disodium, trisodium, tetrasodium, and other higher salts. Such salts can also include noninteger ratios such as the 1.5 sodium salt, the 2.75 sodium, etc. Also, various hydrates including integer and non-integer hydrates, as well as anhydrous forms are contemplated as within the scope of the present invention.

A non-limiting example of the subclass is alendronate monosodium trihydrate.

In other embodiments, other preferred salts are the sodium salt of ibandronate, and risedronate monosodium hemi-pentahydrate (i.e. the 2.5 hydrate of the monosodium salt).

It is recognized that mixtures of two or more of the bisphosphonate actives can be utilized.

The precise dosage of the farnesyl diphosphate synthase inhibiting bisphosphonate will vary with the dosing schedule, the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount of farnesyl diphosphate synthase inhibiting bisphosphonate is chosen to obtain a bone resorption inhibiting effect, i.e. a bone resorption inhibiting amount of the farnesyl diphosphate synthase inhibiting bisphosphonate is administered. For humans, an effective oral dose of farnesyl diphosphate synthase inhibiting bisphosphonate is typically from about 1.5 to about 6000 μg/kg body weight and preferably about 10 to about 2000 μg/kg of body weight.

For the farnesyl diphosphate synthase inhibiting bisphosphonate, alendronate monosodium trihydrate, common human doses which are administered are generally in the range of about 2 mg/day to about 40 mg/day, preferably about 5 mg/day to about 40 mg/day. In the U.S. presently approved dosages for alendronate monosodium trihydrate are 5 mg/day for preventing osteoporosis, 10 mg/day for treating osteoporosis, and 40 mg/day for treating Paget's disease.

In alternative dosing regimens, the farnesyl diphosphate synthase inhibiting bisphosphonate can be administered at intervals other than daily, for example once-weekly dosing, twice-weekly dosing, biweekly dosing, and twice-monthly dosing. In a once weekly dosing regimen, alendronate monosodium trihydrate would be administered at dosages of 35 mg/week or 70 mg/week.

The pharmaceutical compositions herein comprise from about 1 mg to about 100 mg of farnesyl diphosphate synthase inhibiting bisphosphonate, preferably from about 2 mg to 70 mg, and more preferably from about 5 mg to about 70, on a bisphosphonic acid basis. For the bisphosphonate alendronate monosodium trihydrate, the pharmaceutical compositions useful herein comprise about 2.5 mg, 5 mg, 10 mg, 35, mg, 40 mg, or 70 mg of the active on an alendronic acid active weight basis.

Squalene Synthase Inhibitors

The compositions of the present invention comprise a pharmaceutically effective amount of a squalene synthase inhibitor.

The squalene synthase inhibitors of the present invention are useful to block local irritation at injection sites when a farnesyl diphosphate synthase inhibiting bisphosphonate is administered parenterally. Non-limiting examples of squalene synthase inhibitors of the present invention can be categorized into four groups: zaragozic acid/squalestatins, phosphate-derived substrate analogues, carboxylic acid-derived compounds, and quinuclidines and related amines.

Naturally occurring isoforms of the zaragozic acid/squalestatins are characterized by the 2,8-dioxobicyclo[3.2.1]octane-3,4,5-tricarboxylic acid ring system. These naturally occurring forms can be isolated from fungal fermentations and natural products, and also serve as starting materials for diverse semisynthetic and synthetic analogues. Zaragozic acid/squlaestatins of the present invention include compounds described in U.S. Pat. Nos. 5,506,262 and 5,369,125 to Merck & Co., Inc.; JP-7173166 to Takeda; JP-9124655 to Sankyo Co. Ltd.; U.S. Pat. No. 5,430,055 to Pfizer, Inc.; U.S. Pat. No. 5,409,950 to Glaxo Group Ltd.; and JP 9227566 to Sagami Chuo Kagaku Kenkyujo. Non-limiting examples of zaragozic acid/squlaestatins include:

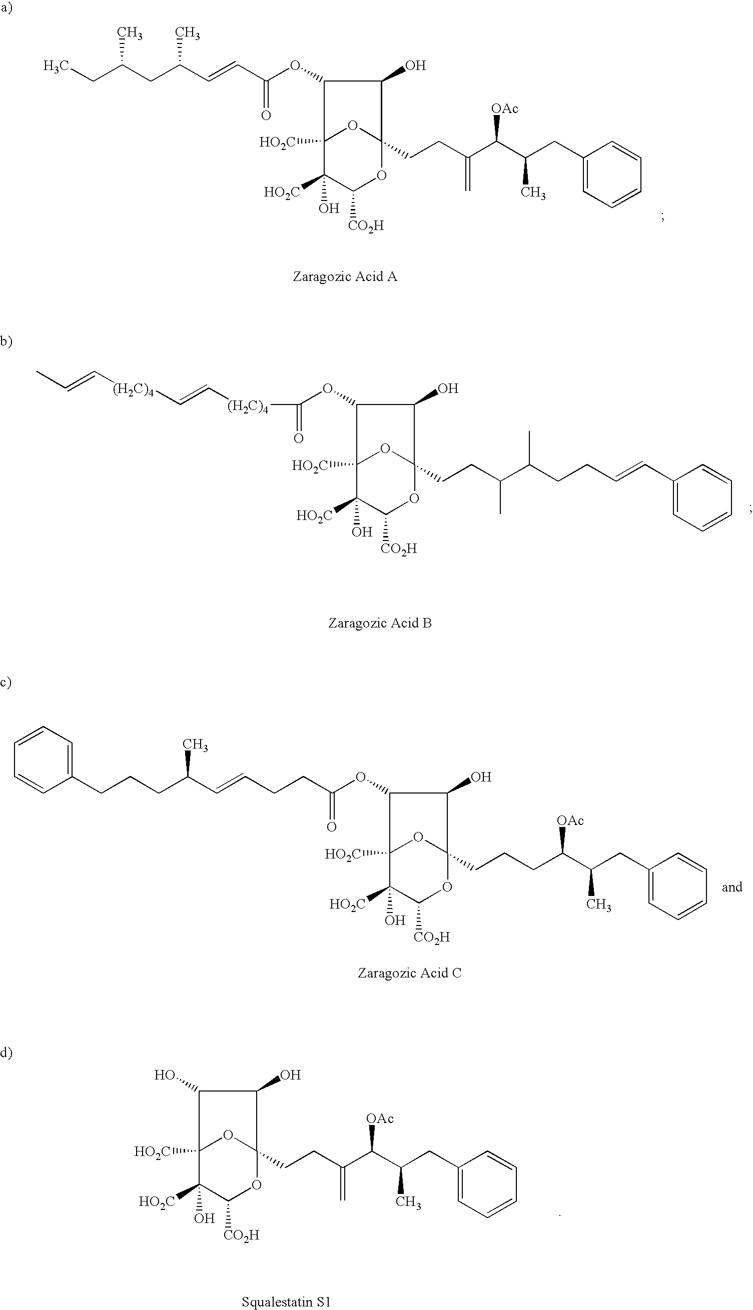

Phosphate-derived inhibitors were originally designed as substrate analogues for squalene synthase, and include compounds described in U.S. Pat. Nos. 5,374,628, 5,428,028, 5,470,845, 5,447,922 to ER Squibb & Sons, Inc. and U.S. Pat. No. 5,441,946 to Rhone-Poulenc Rorer Pharm., Inc. Non-limiting examples of phosphate-derived inhibitors include:

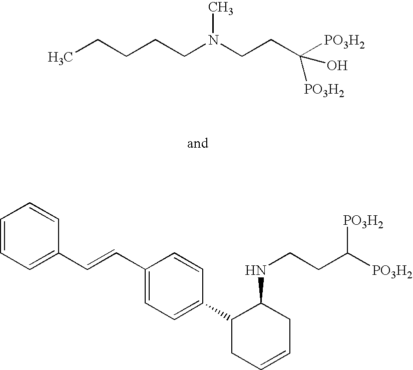

Representative carboxylic acid-derived inhibitors can be characterized as having a lipophilic group(s) couple to one or more carboxylic acid residues. Carboxylic acid-derived residues of the present invention compounds disclosed in JP 7041454 to Sankyo Co. Ltd.; WO 9504025 to Chugai Seiyaku Kabushiki Kaisha; WO 9740006 to Cancer Res. Campaign Tech. Ltd.; JP 7173120 to Banyu; WO 9633159 and WO 9521815 to Abbott Lab; EP 645377, EP 645378 and WO 9521834 to Takeda Chem. Ind. Ltd.; WO 9748701 and EP 814080 to Pfizer, Inc. Non-limiting examples of carboxylic acid-derivatives inhibitors include:

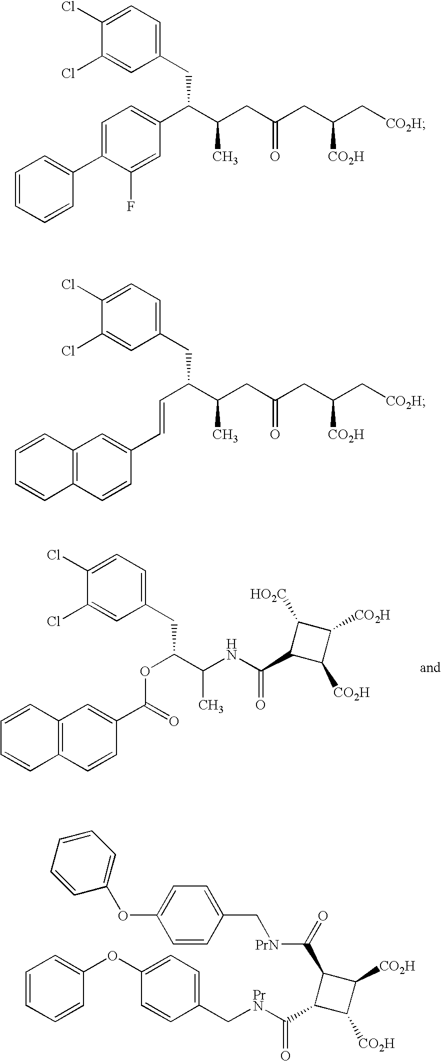

Biaryl quinuclidine, quinuclidine derivatives and related amines are squlaene synthase inhibitors, and include compounds described in WO 9403541, WO 9405660 and WO 9535295 to Zeneca Ltd.; WO 9626938 and JP 8134067 to Yamanouchi Pharm. Co. Ltd.; U.S. Pat. Nos.5,385,912, 5,494,918, 5,395,846, 5,451,596, WO 9531458 and WO 9500146 to Rhone-Poulenc Rorer Pharm., Inc. Nonlimiting examples of quinucidines and related amines include:

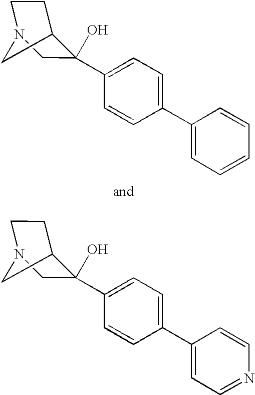

It is recognized that mixtures of two or more of the squalene synthase inhibitors can be utilized.

The precise dosage of the squalene synthase inhibitor will vary with the dosing schedule, the particular compound chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount is chosen to obtain an inhibition of the potentially adverse gastrointestinal effects of the famesyl diphosphate synthase inhibiting bisphosphonate. The amount should be below that level which will inhibit the desired bone resorption inhibiting effect of the nitrogen-containing bisphosphonate. For the squalene synthase inhibitor, human doses which can be administered to humans in the range of about 25 ng/day to about 10 mg/day, preferably from about 25 nanograms to about 1 milligram, although other ranges can be used. A nonlimiting exemplary dose is about 1 microgram, for a human subject. In a once-weekly dosing, the squalene synthase inhibitor can be administered to humans in the range of about 25 ng/dose to about 10 mg/dose, preferably from about 25 nanograms to about 1 milligram, although other ranges can be used. A nonlimiting exemplary dose is about 1 microgram, for a human subject.

Other Components of the Pharmaceutical Compositions

The famesyl diphosphate synthase inhibiting bisphosphonate and the squalene synthase inhibitor are typically administered in admixture with suitable pharmaceutical diluents, excipients, or carriers, collectively referred to herein as “carrier materials”, suitably selected with respect to parenteral administration, consistent with conventional pharmaceutical practices.

For parenteral dosing, in particular, subcutaneous administration, the agents are typically combined in aqueous vehicle, such as sterile water or sterile isotonic (0.9%) sodium chloride.

The compounds used in the present method can also be coupled with soluble polymers as targetable drug carriers. Such polymers can include polyvinylpyrrolidone, pyran copolymer, polyhydroxylpropyl-methacrylamide, and the like.

Methods of the Present Invention

The present invention comprises methods for treating abnormal bone resorption in mammals. The present invention also comprises methods for preventing abnormal bone resorption in mammals. In preferred embodiments of the present invention, the mammal is a human.

The methods and compositions of the present invention are useful for both treating and preventing abnormal bone resorption and conditions associated therewith. Conditions associated with abnormal bone resorption include both generalized and localized bone loss. Also, the creation of bone having an abnormal structure, as in Paget's disease, can be associated with abnormal bone resorption. The term “generalized bone loss” means bone loss at multiple skeletal sites or throughout the skeletal system. The term “localized bone loss” means bone loss at one or more specific, defined skeletal sites.

Generalized boss loss is often associated with osteoporosis. Osteoporosis is most common in post-menopausal women, wherein estrogen production has been greatly diminished. However, osteoporosis can also be steroid-induced and has been observed in males due to age. Osteoporosis can be induced by disease, e.g., rheumatoid arthritis, it can be induced by secondary causes, e.g., glucocorticoid therapy, or it can come about with no identifiable cause, i.e. idiopathic osteoporosis. In the present invention, preferred methods include the treatment or prevention of abnormal bone resorption in osteoporotic humans.

Localized bone loss has been associated with periodontal disease, with bone fractures, and with periprosthetic osteolysis (in other words where bone resorption has occurred in proximity to a prosthetic implant).

Generalized or localized bone loss can occur from disuse, which is often a problem for those confined to a bed or a wheelchair, or for those who have an immobilized limb set in a cast or in traction.

The methods and compositions of the present invention are useful for treating and or preventing the following conditions or disease states: osteoporosis, which can include post-menopausal osteoporosis, corticosteroid-induced osteoporosis, male osteoporosis, disease-induced osteoporosis, idiopathic osteoporosis; Paget's disease; abnormally increased bone turnover; osteomalacia; periodontal disease; localized bone loss associated with periprosthetic osteolysis; and bone fractures.

The compositions and methods of the present invention are administered and carried out until the desired therapeutic effect is achieved.

In the methods of the present invention the famesyl diphosphate synthase inhibiting bisphosphonate and the squalene synthase inhibitor are generally administered concurrently. In alternate embodiments, the farnesyl diphosphate synthase inhibiting bisphosphonate and the squalene synthase inhibitor can be adminsitered sequentially.

The following Examples are presented to better illustrate the invention.

### EXAMPLE 1

Squalene Synthase Inhibitor Suppression of the Inhibition of Protein Isoprenylation Induced in Ch1.Es Esophageal Fibroblasts by ALN

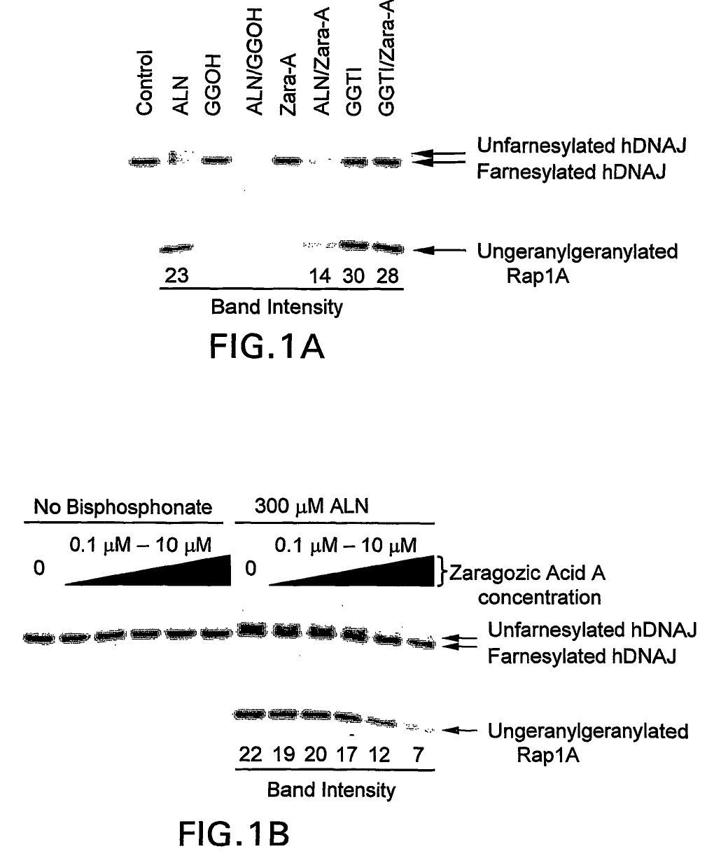

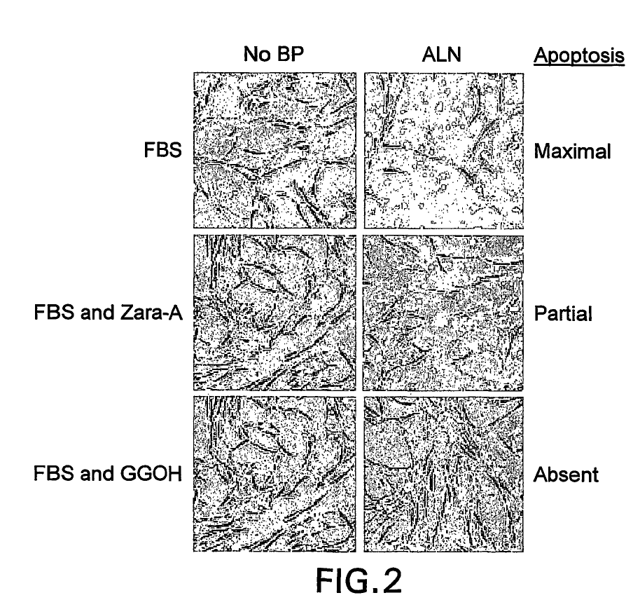

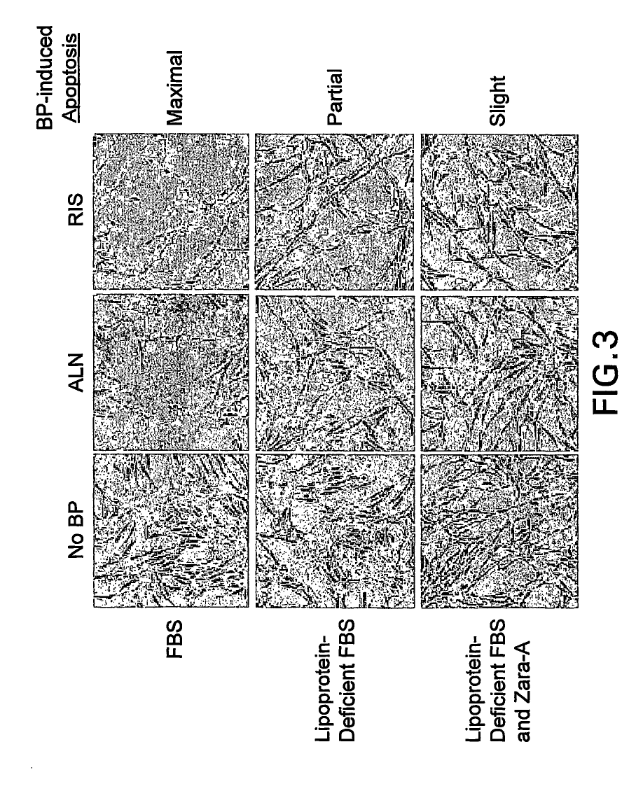

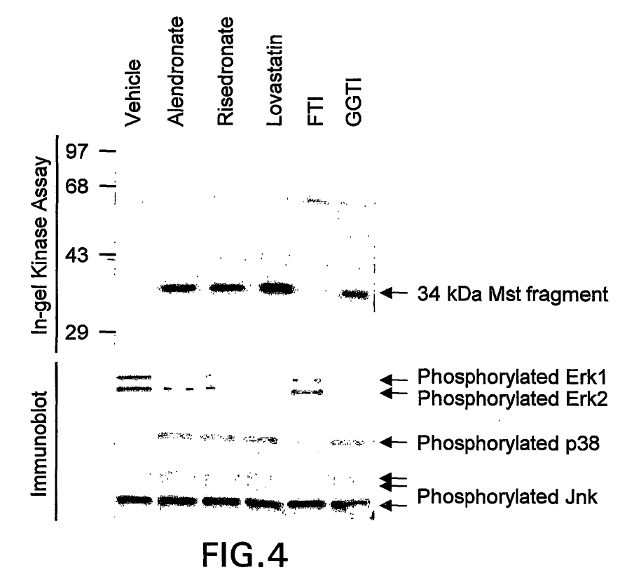

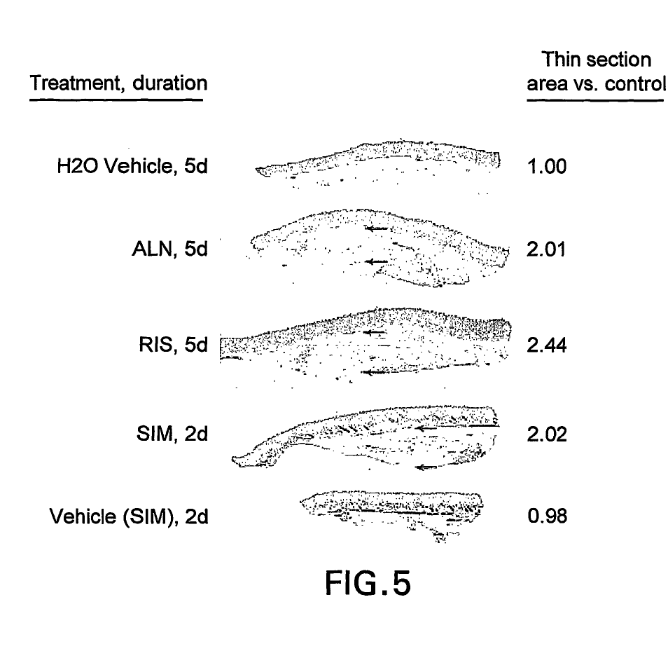

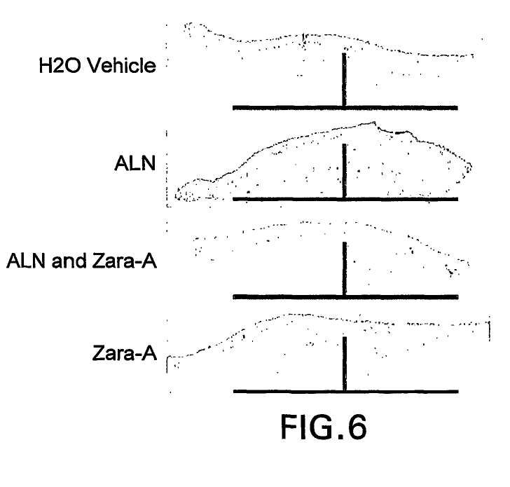

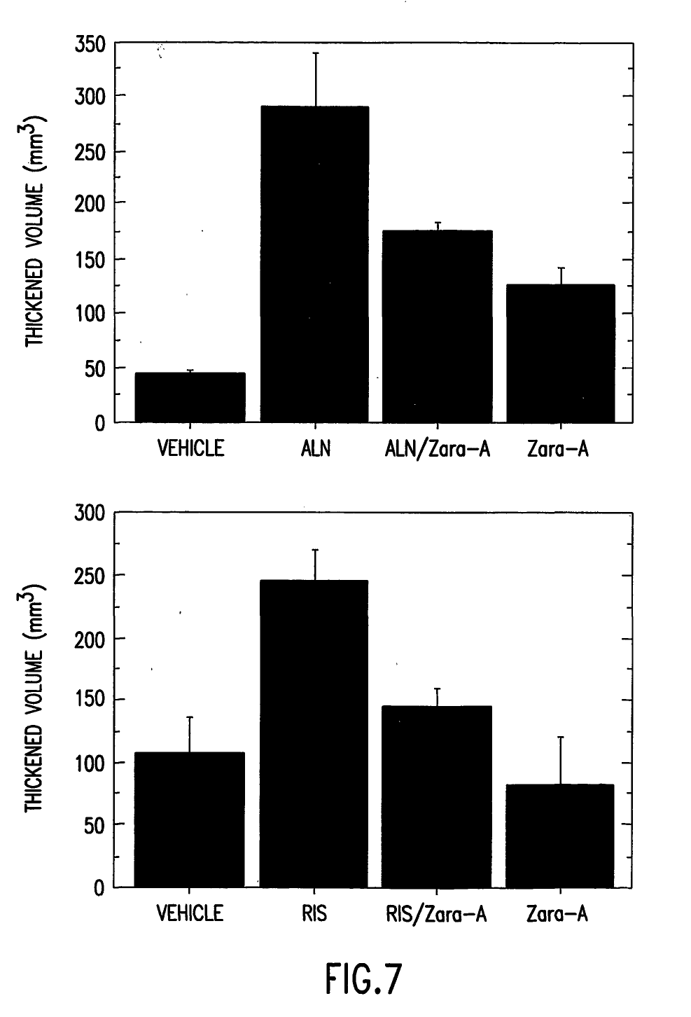

Ch1.Es fibroblasts were grown in culture for 24 hours prior to treatment with alendronate [ALN] at 300 μM. Cells were treated for 24 hours and protein lysates were analyzed for suppression of protein isoprenylation. Markers included either anti-hDNAJ, a farnesylated protein that migrates more slowly when farnesylation is absent (two bands observed) or anti-Rap1A, which we have shown previously to bind preferentially to Rap1A if isoprenylation is blocked (presence of a band indicates absence of protein geranylgeranylation. See FIG. 1.

(A) Cells pretreated for 24 hours with nothing or the squalene synthase inhibitor, Zaragozic Acid A (Zara-A) at 3 μM (three lanes indicated). This was continued for an additional 24 hours in the presence or absence of ALN, geranylgeranyl transferase inhibitor (GGTI, 100 nM) or geranylgeraniol (GGOH), as indicated. Note that ALN, but not GGTI, suppression of geranylgeranylation is reduced by the inclusion of Zara-A.

(B) Cells were pretreated for 24 hours with varying concentrations of Zara-A (0 and 0.1-10 μM) and then continued for another 24 hours in the absence or presence of ALN. Note that in the absence of Zara-A, ALN strongly inhibits both farnesylation and geranylgeranylation. Zara-A dose-dependently reduces the ALN effect.

In (A) and (B) the Zara-A acts as a molecular trap by preventing use of farnesyl diphosphate stores for cholesterol synthesis. Pools of farnesyl diphosphate and its derivative, geranylgeranyl diphosphate, are thus shunted into the protein isoprenylation pathway even after ALN has been added to the cells.

### EXAMPLE 2

As a specific embodiment of the present invention, 0.5 mg of a farnesyl diphosphate synthase inhibiting bisphosphonate or a pharmaceutically acceptable salt thereof and 5 micrograms of a squalene synthase inhibitor are dissolved in sterile isotonic (0.9%) sodium chloride to a total volume of 0.5 mL.

What is claimed is:

1. A pharmaceutical composition comprising a farnesyl diphosphate synthase inhibiting bisphosphonate or a pharmaceutically acceptable salt thereof and a squalene synthase inhibitor.

2. The pharmaceutical composition according to claim 1 wherein said farnesyl diphosphate synthase inhibiting bisphosphonate is selected from the group consisting of alendronate, ibandronate, incadronate, minodronate, neridronate, olpadronate, risedronate, piridronate, pamidronate, zolendronate, pharmaceutically acceptable salts thereof, and mixtures thereof.

3. The pharmaceutical composition according to claim 2 wherein said squalene synthase inhibitor is selected from the group consisting of: 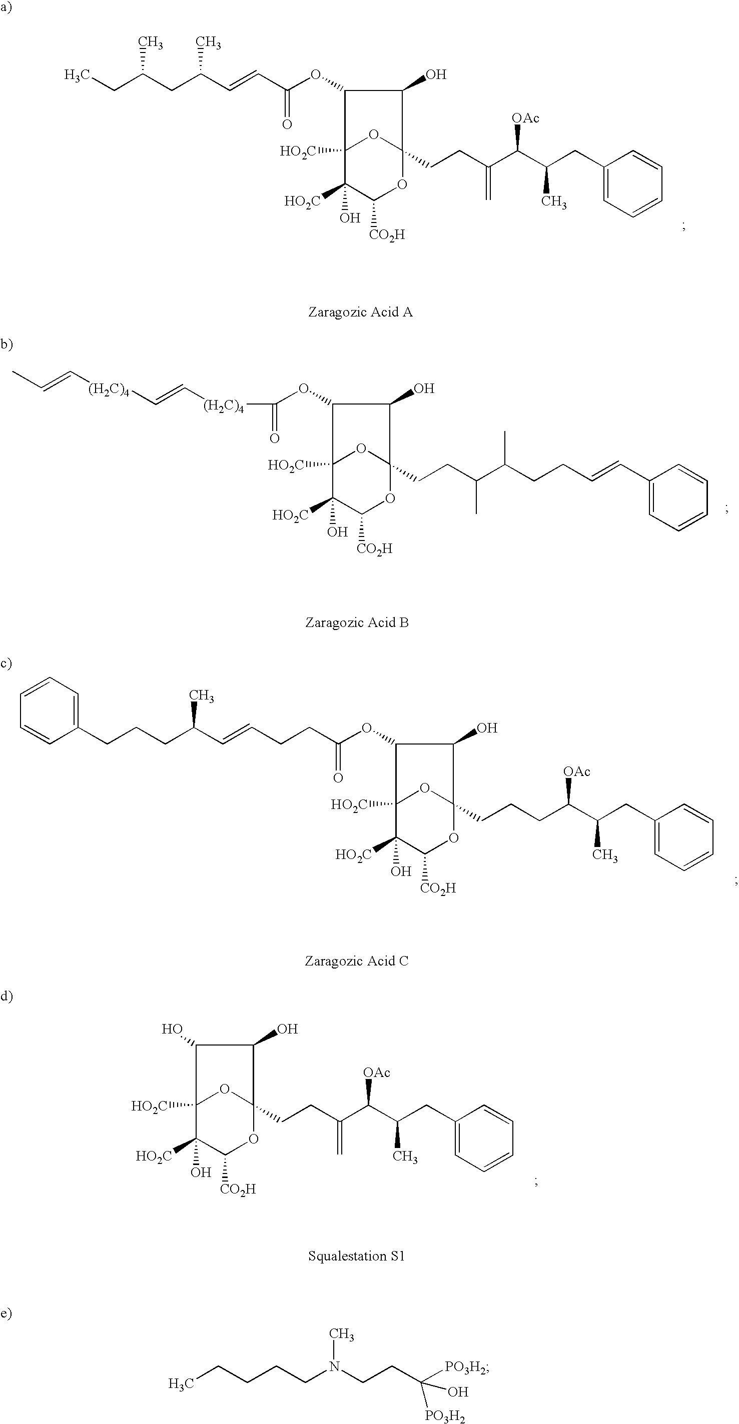 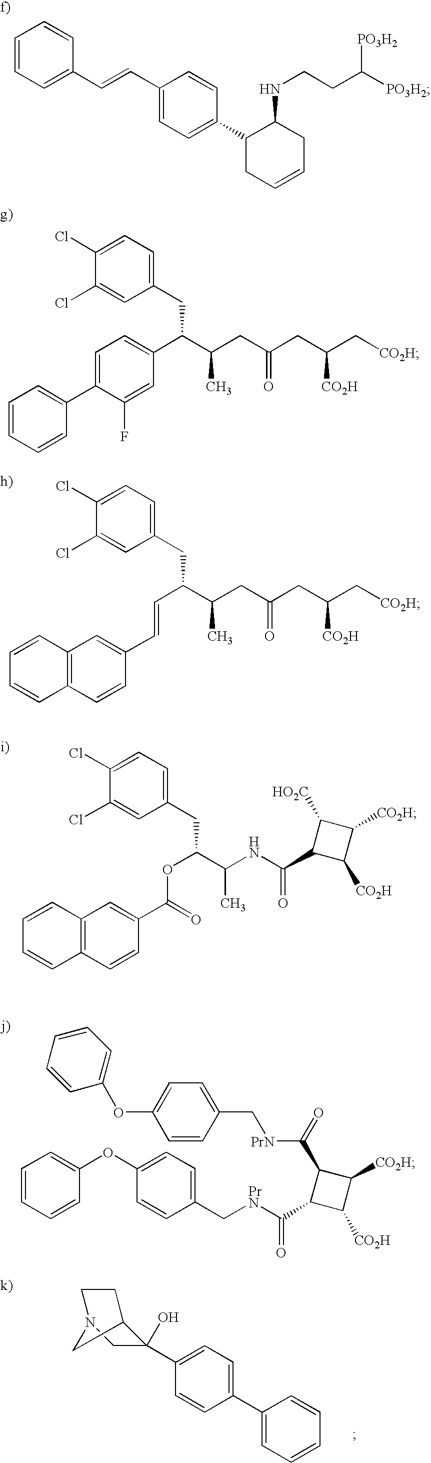 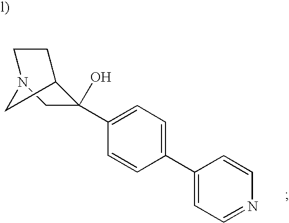
and the pharmaceutically acceptable salts and mixtures thereof.

4. The pharmaceutical composition according to claim 3 wherein said squalene synthase inhibitor is selected from the group consisting of: 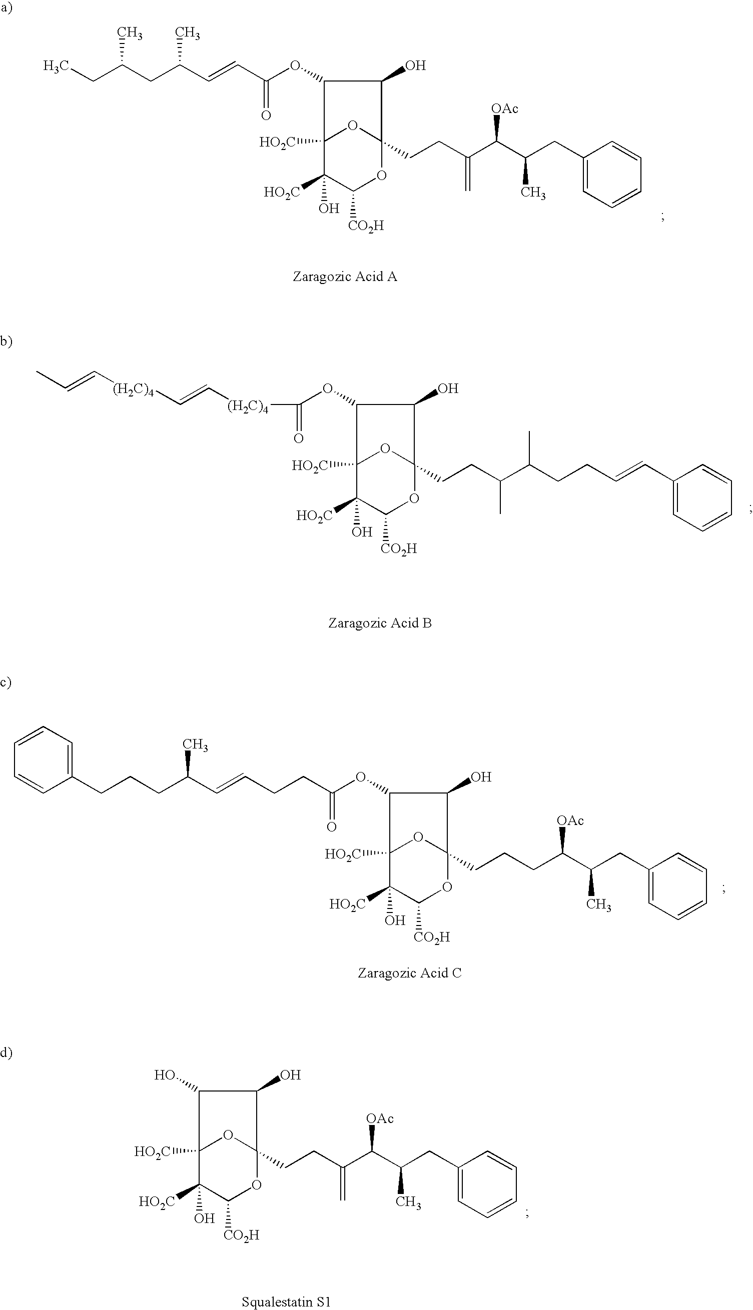
and the pharmaceutically acceptable salts and mixtures thereof.

5. The pharmaceutical composition according to claim 4 wherein said squalene synthase inhibitor is zaragozic acid A and the pharmaceutically acceptable salts thereof.

6. The pharmaceutical composition according to claim 5 wherein said farnesyl diphosphate synthase inhibiting bisphosphonate is alendronate and pharmaceutically acceptable salts thereof.

7. The pharmaceutical composition according to claim 6 wherein said farnesyl diphosphate synthase inhibiting bisphosphonate is alendronate monosodium trihydrate.

8. A pharmaceutical composition comprising from 1 to 100 mg of a famesyl diphosphate synthase inhibiting bisphosphonate or a pharmaceutically-acceptable salt thereof and from 25 nanograms to 1 milligram of a squalene synthase inhibitor.

9. The pharmaceutical composition according to claim 8 comprising from 2 to 70 mg of a farnesyl diphosphate synthase inhibiting bisphosphonate or a pharmaceutically-acceptable salt thereof and from 1 microgram to 1 milligram of a squalene synthase inhibitor.

10. A pharmaceutical composition comprising from 1 to 100 mg of alendronate monosodium trihydrate, on an alendronic acid weight basis, and from 1 microgram to 1 milligram of zaragozic acid A.

11. The pharmaceutical composition according to claim 10 comprising from about 2 to about 70 mg of alendronate monosodium trihydrate, on an alendronic acid weight basis, and from about and from about 1 microgram to about 1 milligram of zaragozic acid A.

12. The pharmaceutical composition which is prepared by combining a farnesyl diphosphate synthase inhibiting bisphosphonate and a squalene synthase inhibitor.

13. A method for inhibiting bone resorption in a mammal in need thereof comprising administering a farnesyl diphosphate synthase inhibiting bisphosphonate or pharmaceutically acceptable salt thereof and a squalene synthase inhibitor.

14. The method according to claim 13 wherein said mammal is a human.

15. A parenteral method for treating or preventing osteoporosis or Paget's disease in a mammal in need thereof comprising administering a farnesyl diphosphate synthase inhibiting bisphosphonate or pharmaceutically acceptable salt thereof and a squalene synthase inhibitor.

16. A method according to claim 15 wherein said mammal is a human.

17. A method according to claim 16 wherein said farnesyl diphosphate synthase inhibiting bisphosphonate or pharmaceutically-acceptable salt thereof is selected from the group consisting of alendronate, ibandronate, incadronate, minodronate, neridronate, olpandronate, risedronate, piridronate, pamidronate, zolendronate, pharmaceutically acceptable salts thereof, and mixtures thereof.

18. A method according to claim 16 wherein said squalene synthase inhibitor is selected from the group consisting zaragozic acid A, zaragozic acid B, zaragozic acid C, squalestatin S, and pharmaceutically acceptable salts and mixtures thereof.

19. A method for inhibiting bone resorption in a mammal in need thereof comprising sequentially administering a squalene synthase inhibitor and a farnesyl diphosphate synthase inhibiting bisphosphonate or pharmaceutically acceptable salt thereof.
